(19) United States
(12) Patent Application Publication
(10) Pub. No.: US 2005/0165222 A1
(43) Pub. Date: Jul. 28, 2005

(54) Method of reducing leachate from protein a affinity media

(75) Inventors: Thomas D. Leete, Westford, MA (US); Theresa S. Creasey, Bedford, MA (US); Robert M. Smith, Stow, MA (US); James M. Coull, Westford, MA (US); Darryl J. Pappin, Boxborough, MA (US); Brooks Edwards, Cambridge, MA (US); Mark A. McCoy, Framingham, MA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 10/966,188

(22) Filed: Oct. 15, 2004

Related U.S. Application Data
(60) Provisional application No. 60/511,521, filed on Oct. 15, 2003.

(51) Int. Cl. C07K 16/18; C12N 9/99

(52) U.S. Cl. 530/387.1; 435/184

(57) ABSTRACT

Disclosed are methods and compositions that may be used for purifying antibodies.

### CROSS REFERENCE TO RELATED APPLICATION

This application claims a priority benefit of U.S. Patent Application No. 60/511,521, filed Oct. 15, 2003, which is incorporated herein by reference.

### INTRODUCTION

Protein A affinity chromatography is a conventional means for purifying polyclonal and monoclonal antibodies. Typically, an antibody-containing sample is adsorbed onto a protein A support under neutral or basic conditions (e.g., pH 6 to 9), and the support is washed with the same buffer (or optionally with different buffers) to elute non-antibody proteins and other impurities. After the impurities have been eluted, the adsorbed antibodies can be eluted in purified form using an acidic buffer (e.g., having a pH<6.5).

A common problem in protein A-mediated purifications is that protein A or antibody-protein A complexes (collectively referred to as “protein A leachate”) can be released from the support and can coelute with the purified antibodies during the acidic elution step. This protein A leachate can be problematic for a number of reasons. For example, with in vivo administration of antibody, protein A contaminants can alter patient response, interfere with the interpretation of diagnostic results or act as an immunomodulator affecting a variety of immunological phenomena. Furthermore, in some cases, protein A leachate has proven toxic in clinical trials, see for example, Bensinger, W., et. al. Journal of Biological Response Modifiers, v. 3, 347 (1984); Messerschmidt, et. al. Journal of Biological Response Modifiers, v. 3, 325 (1984); Terman D., et. al. European Journal of Cancer & Clinical Oncology, v. 21, 1105 (1985); and Ventura, G. Cancer Treatment Reports, v. 71, 411 (1987). As a result, assays have been developed to monitor protein A leachate, for example P. Gagnon. (1996) Purification Tools for Monoclonal Antibodies, Validated Biosystems, Tuscon; and G. Sofer, et al. (1991) Process Chromatography, A Guide to Validation, Academic Press, San Diego.

Rather than monitoring leachate levels, which requires determination of leachate thresholds and the validation of monitoring methods, it has become common to remove the protein A leachate. However, this is not ideal since removal of protein A leachate requires further purification steps and additional expense.

Accordingly, there is a need to reduce the amount of protein A leachate from protein A affinity media.

Non-Limiting Summary

The present application relates to methods of reducing protein A leachate levels from protein A chromatography columns and to methods of purifying antibodies. In addition, the present application relates to protein A affinity chromatography binding buffer compositions and to antibody compositions.

In some embodiments, methods are provided for purifying antibody-containing samples. In some embodiments, an antibody sample is contacted with a protein A affinity support under conditions such that antibodies are captured by binding to protein A on the support to form support-bound antibodies. Non-antibody components may then be removed from the support bound antibodies, and the support-bound antibodies may then be released from the support to obtain a purified antibody preparation. Prior to or during the contact of the antibody sample with the support, the sample can be contacted with at least one protease inhibitor in an amount effective to reduce the level of protein A leachate in the purified antibody preparation relative to the level of protein A leachate that is present in the purified antibody preparation when the at least one protease inhibitor is not contacted with the sample.

In some embodiments, the at least one protease inhibitor comprises a metalloproteinase inhibitor. In some embodiments, the at least one protease inhibitor comprises a metal chelator. In some embodiments, the at least one protease inhibitor comprises ethylenediamine tetraacetic acid (EDTA).

In some embodiments, the at least one protease inhibitor comprises a serine protease inhibitor. In some embodiments, the at least one protease inhibitor comprises an inhibitor of at least one of trypsin, chymotrypsin, plasmin, plasma kallikrein, thrombin, clotting factors, tissue proteinases, leukocytic proteinases, elastase-like serine protease and urokinase. In some embodiments, the at least one protease inhibitor comprises an inhibitor of at least one of trypsin, chymotrypsin, plasmin, plasma kallikrein and thrombin. In some embodiments, the at least one protease inhibitor comprises a benzenesulfonyl fluoride compound. In some embodiments, the at least one protease inhibitor comprises at least two different serine protease inhibitors. In some embodiments, the at least two different serine protease inhibitors are inhibitors of at least two of trypsin, chymotrypsin, plasmin, plasma kallikrein, thrombin, clotting factors, tissue proteinases, leukocytic proteinases, elastase-like serine protease and urokinase. In some embodiments, the at least two different serine protease inhibitors are inhibitors of at least two of trypsin, chymotrypsin, plasmin, plasma kallikrein and thrombin. In some embodiments, the at least one protease inhibitor comprises a metalloproteinase inhibitor and a serine protease inhibitor, such as a metal chelator, e.g., EDTA.

In some embodiments, such as discussed above or further below, the at least one protease inhibitor is provided in an amount effective to reduce the level of protein A leachate in the purified antibody preparation by at least 50%, or by at least 75%, or by at least 90%, relative to the level of protein A leachate that is present in the purified antibody preparation when the at least one protease inhibitor is not contacted with the sample.

In some embodiments, the protein A affinity support is provided in a chromatography column. Non-antibody components may be removed, for example, by passing a buffer through the support under conditions such that support bound antibodies are retained on the support.

The antibodies that are purified may be any type of antibody or mixture of antibodies. In some embodiments, the antibody sample may comprise one or more monoclonal antibodies, one or more monoclonal antibody fragments, one or more polyclonal antibodies, or one or more polyclonal antibody fragments. In some embodiments, the sample comprises an IgG antibody or IgG antibody fragment. In some embodiments, the sample comprises a human antibody or human antibody fragment. In some embodiments, the sample comprises a human IgG antibody or human IgG antibody fragment. In some embodiments, the sample comprises serum or ascites or is obtained from serum, ascites, or tissue culture. In some embodiments, the sample comprises or is derived from human blood.

These and other embodiments of the present teachings will become more fully apparent in light of the following description.

### DETAILED DESCRIPTION

As noted above, the present application provides methods and compositions that may be used for antibody purification by protein A-based affinity techniques. In particular, methods are provided for reducing the level of protein A leachate in such affinity-purified antibody preparations.

The antibody-containing sample to be purified in accordance with the teachings of the present application may comprise any antibodies or antibody fragments that can be captured by support-bound protein A. Without being bound by any theory, protein A is believed to form a high affinity complex with antibodies by binding noncovalently to the Fc region of antibodies such as IgG antibodies. Thus, antibodies or antibody fragments that contain an Fc region or related motif are expected to bind to protein A and can be immobilized on protein A affinity supports. Antibodies may have any of a variety of forms, such as polyclonal antibodies, monoclonal antibodies, humanized antibodies, single-chain antibodies, and fragments thereof. Typically, antibodies will also include an antigen-specific region or regions which confer antigen-binding specificity that may be advantageous for purposes of therapy, antigen-purification, and diagnostics, for example.

Typically, monoclonal antibodies may be characterized as having a substantially homogeneous antibody population, (i.e. the individuals of the antibody population are identical except for naturally occurring mutations) and have substantially similar binding affinity and specificity. Monoclonal antibodies can be prepared by a large variety of methods and can be derived from any of a large variety of mammalian species such as mouse, rat, hamster, guinea pig, rabbit, sheep, goat, human, cow, cat, dog, horse and pig.

Monoclonal antibodies have usually been prepared using hybridoma technologies pioneered by Kohler and Milstein in the 1970's (e.g., see Kohler et al., Nature, 256, 495-97 (1975)). For example, following immunization of a mammal species with an antigen, the spleen of the animal can be removed and converted into a whole cell preparation. The immune cells from the spleen cell preparation can be fused with myeloma cells to produce hybridomas. The hybridomas may be cultured, and the culture fluid may be tested against the antigen to facilitate isolation of hybridoma cultures that produce monoclonal antibodies specific for the antigen. Introduction of the hybridoma into the peritoneum of the host species produces a peritoneal growth of the hybridoma. Collection of the ascites fluid yields body fluid containing the monoclonal antibody. Also, cell culture supernatant from the hybridoma cell culture can be used. Monoclonal antibodies can also be produced, for example, using murine-derived hybrid cell line wherein the antibody is an IgG or IgM type immunoglobulin. Chimeric and recombinant monoclonal antibodies (or truncated forms of antibodies) can also be prepared by recombinant DNA techniques and expressed using optimized host cells. Monoclonal antibodies can be employed in various diagnostic and therapeutic compositions and methods, including but not limited to passive immunization and anti-idiotype vaccine preparation.

Polyclonal antibodies typically comprise a heterogeneous population of different antibodies derived from multiple clones, each of which is specific for one of a number of determinants found on an antigen. Usually, to make polyclonal antibodies, a whole pathogen, an isolated antigen, or an antigen or epitope that is coupled to a carrier, is introduced by inoculation or infection into a host that induces the host to make antibodies against the pathogen or antigen. Crude polyclonal antibody sera can be produced by any method known to those of skill in the art. Antigen-containing culture fluid or inoculum can be administered with a stimulating adjuvant to a mammal. After repeated challenge with antigen, portions of blood serum can be removed and further purified if desired.

The protein A affinity support can be any support that is capable of binding antibodies with high affinity, and preferably capable of binding a broad spectrum of antibodies independent of antigen specificity. The protein A affinity support can be prepared by any appropriate method. A variety of support materials have been employed for protein A affinity columns and are commercially available, such polystyrene/divinylbenzene (e.g., Poros® A/M, Poros® 50 A, and Poros® A LP available from Applied Biosystems, Foster City, Calif.), controlled-pore glass (e.g., Prosep™ from Bioprocessing, Consett, County Durham, UK), cross-linked agarose (e.g., Sepharose™ A Fast Flow from Amersham, Uppsala, Sweden), and expanded bed (e.g., Streamline™ A from Amersham, Uppsala, Sweden) (see also the 2000-2001 or current Biochemicals and Reagents catalog from Sigma Aldrich for other protein A and protein A affinity support products). Moreover, protein A affinity supports can be prepared by any of a variety of methods for attaching proteins to support materials (e.g., see G. T. Hermanson, Bioconjugate Techniques, Academic Press, San Diego, Calif., 1996, particularly Chapter 15 entitled “Modification with Synthetic Polymers”, and Chemistry of Protein Coniugation and Cross-Linking, S. S. Wong, CRC Press, Boca Raton, Fla., 1993, particularly Chapter 12 entitled “Conjugation of Proteins to Solid Matrices”). Typically, the support contains functional groups such as carboxyl or amino groups that are suitable for coupling to complementary functional groups that are present in protein A. For example, protein A can be coupled to a support using a carbodiimide or N,N′-carbonyldiimidazole catalyst to couple amino groups to carboxyl groups. Various other coupling techniques, such as amide formation by reaction of amines with activated carboxyl groups such as N-succinimidyl carboxylate esters, disulfide formation, reaction of amines or thiols with epoxides, thioether formation by reacting a thiol with a maleimide, and the like may also be suitable. Protein A may also be coupled to a support via a linker molecule to help separate the support surface from the protein A molecule (e.g., see Hermanson and Wong, supra).

In some embodiments, the protein A support is provided in a chromatography column, and purification of antibodies is facilitated by flowing sample and buffers through the column bed to wash the column or elute the antibodies of interest. In other embodiments, the protein A support may be used as a powder or solid that is added to the sample under conditions that allow sample antibodies to adhere to the protein A. Unbound sample components can be removed from the support by decanting the surrounding solution (or by removing a supernatant after the support has been centrifuged to the bottom of a container). The support can be washed with one or more aliquots of one or more wash buffers to further remove non-bound sample components (with the help of centrifugation or gravity-mediated sedimentation), followed by the addition of elution buffer to remove a purified antibody preparation from the support for further analysis or other uses.

The sample may be any sample that contains one or more antibodies that are to be purified. Suitable sources include, for example, serum samples (or samples that are derived from serum) from mouse, rat, hamster, guinea pig, rabbit, sheep, goat, human, cow, cat, dog, horse or pig, etc.; tissue culture samples; ascites samples from a mouse, rat, hamster, guinea pig, rabbit, sheep, goat, human, cow, cat, dog, horse or pig; synthetically prepared antibodies; recombinantly produced antibodies; or pre-purified antibodies; any of which may be obtained from commercial or non-commercial sources. Optionally an antibody sample can be diluted with from, for example, 1 to 1000 parts, 1 to 100 parts, or 1 to 50 parts of a buffer to facilitate binding of the antibodies to the protein A support. It will be understood that the above ranges can include all ranges set by the integers from 1 to 1000.

Prior to or during the step in which the antibody sample is contacted with the protein A affinity support, the sample is contacted with at least one protease inhibitor in an amount effective to reduce the level of protein A leachate in the purified antibody preparation relative to the level of protein A leachate that is present in the purified antibody preparation when the at least one protease inhibitor is not contacted with the sample. The protease inhibitor(s) may be contacted with the antibody sample in any suitable way. For example, the protease inhibitor(s) can be added as a powder, as a concentrated stock solution, or by diluting the sample with buffer that contains inhibitor(s). The antibody sample may be contacted with the one or more inhibitors for any appropriate amount of time, although inhibition is usually complete within a few minutes or seconds. It may be preferable to contact the antibody sample with the one or more inhibitors before the antibody sample is contacted with the protein A affinity support, to help reduce the generation of protein A leachate.

As used herein, the terms “protease inhibitor” and “protease inhibitor cocktail” refer to any molecule or collection of molecules that are capable of interfering with the proteolytic activity of one or more proteases that may be present in the antibody sample and that cause the release of protein A leachate from the protein A affinity support. The inhibitors may be capable of inhibiting any of a large variety of proteases as are known or unknown in the art, provided that the one or more inhibitors are individually or collectively able to reduce the generation of protein A leachate. For example, the one or more inhibitors may be capable of inhibiting serine proteases, cysteine proteases, metalloproteases and aspartic proteases. Specific examples of proteases that may be inhibited include, but are not limited to, trypsin, chymotrypsin, thrombin, plasmin, papain, plasma kallikrein, clotting factors such as protease factor IXa, protease factor Xa, protease factor Xia, protease factor XIIa, tissue proteinases, leukocytic proteinases, elastase-like serine protease, urokinase, calpain, elastase, cathepsin G, cathepsin B, cathepsin L, endoproteinase Glu-C, pepsin, renin, chymosin, bromelain, and ficin.

Inhibitor cocktails (comprising two or more different protease inhibitors) may also be employed. Such protease inhibitor cocktails may include two or more inhibitors selected from, but not limited to, serine protease inhibitors, cysteine protease inhibitors, metalloprotease inhibitors and aspartic protease inhibitors. In some embodiments, the two or more inhibitors may be selected from inhibitors of trypsin, chymotrypsin, plasmin, plasma kallikrein, thrombin, clotting factors, tissue proteinases, leukocytic proteinases, elastase-like serine protease and urokinase.

Exemplary inhibitors or inhibitor cocktails include, for example, sulfonyl fluoride compounds such as PMSF (phenylmethylsulfonyl fluoride, available from Sigma Aldrich, and typically added as a stock solution in isopropranol, ethanol, or methanol), benzenesulfonyl fluoride compounds (which are also sulfonyl fluoride compounds) such as 4-(2-aminoethyl)benzenesulfonylfluoride HCl (sold commercially as Pefabloc® SC by Roche Diagnostics), and Protease Inhibitor Cocktail Set III (available from Calbiochem as a cocktail of six protease inhibitors that inhibit aspartic, cysteine, and serine proteases and aminopeptidases, namely 100 mM AEBSF, HCl, 80 mM aprotinin, 5 mM bestatin, 1.5 mM E-64, 2 mM leupeptin hemisulfate, and 1 mM Pepstatin A, all in DMSO). Additional protease inhibitors have been described in the literature and/or are available from various commercial sources (see for example the 2000-2001 or current Biochemicals and Reagents catalog from Sigma Aldrich under “Protease Inhibitors” and “Protease and Phosphatase Inhibitor Cocktails).

In some embodiments, the at least one protease inhibitor comprises a metallo-proteinase inhibitor, such as a metal chelator (e.g., EDTA). In some embodiments, the at least one protease inhibitor comprises a serine protease inhibitor. In some embodiments, the at least one protease inhibitor comprises at least one metalloproteinase inhibitor and at least one serine protease inhibitor, such as described herein. In some embodiments, the at least one protease inhibitor comprises at least EDTA and at least one serine protease inhibitor. In some embodiments, the at least one protease inhibitor comprises at least one metallo-proteinase inhibitor, such as a metal chelator, and at least one trypsin inhibitor. In some embodiments, the at least one protease inhibitor comprises at least EDTA and at least one trypsin inhibitor.

The term “buffer” refers to any buffer known to those of skill in the art for use in conjunction with the present teachings. Exemplary buffer types that may be useful herein include “binding buffers”, “washing buffers”, “elution buffers” and “neutralization buffers”, for example, and may include, but are not limited to, any of the Good buffers found in, for example, N. E. Good et. al. Biochemistry, 5: 467 (1966); N. E. Good et. al. Meth. Enzymol., v. 24, Part B, p. 53 (1972), W. J. Fergeson et. al. Anal. Biochem. 104: 300 (1980); and the 2000-2001 or current Biochemicals and Reagents catalog from Sigma Aldrich. Examples of specific buffers include, but are not limited to, glycine/NaOH buffers, borate buffers, phosphate buffers.

For loading (also referred to as “adhering” or “adsorbing”) protein A onto the protein A affinity support, the antibody sample can be diluted, dialyzed, or reconstituted with a binding buffer that facilitates loading of antibodies onto the support. Such binding buffers can be selected, for example, from one or more of glycine/NaOH buffer, borate buffer or phosphate buffer, typically having a pH in the range of 6.0 to 9.0 or 7.0 to 9.0, although pH values outside these ranges may also be suitable. Representative stock solutions of binding buffers include, but are not limited to, 1-1.5 M glycine/NaOH in 2-3 M NaCl, 1-1.5 M sodium borate in 2-3 M NaCl, and 10-100 mM sodium phosphate and 0.1-0.2 M NaCl. Typically, final buffer concentrations range from about 20 mM to about 200 mM, although concentrations outside this range may also be used.

The antibody sample can be contacted with the protein A affinity support for a time sufficient to adsorb the desired amount of antibody to the support. In column chromatography embodiments, the antibody sample is loaded at a flow rate and antibody concentration selected to ensure sufficient antibody binding to the support. Such conditions can easily be developed by routine optimization (e.g., see R. L. Fahrner et al., Biotechnol. Appl. Biochem 30: 121-128 (1999)). Similar considerations apply to embodiments wherein the antibody sample is contacted with free protein A affinity support in a vessel (batch mode).

Non-antibody sample components can optionally be removed by washing the support with one or more washing buffers which may be the same as or different from the binding buffer. For example, washing buffers may comprise any of the Good buffers mentioned above, usually having a pH in the range of 6.0-9.0 or 7.0-9.0.

After the optional washing of the affinity column, the bound antibody can be removed from the support using an elution buffer. Eluting buffers can be selected from, for example, citrate buffer, a glycine/HCl buffer or a phosphate buffer, such that the pH is acidic. For example, in some embodiments, the pH of the elution buffer is from 2.5-6.5, or is less than 5, or is less than 4, or is less than 3, or is less than 2.5. Exemplary buffers may include 0.1 M sodium citrate, 0.1-0.2 M glycine/HCl, 0.1 M sodium phosphate and aqueous acetic acid (e.g., 75 mM acetic acid).

Finally, the purified antibody can optionally be returned to a more neutral pH using base (e.g., KOH or NaOH) or a neutralization buffer, to increase the pH, usually to 7 or greater. Neutralization buffers can include but are not limited to any of the Good buffers having a pH in the range of 7.0-9.0.

According to some embodiments, contact of the antibody sample with the one or more protease inhibitors is performed before the sample is contacted with the protein A affinity support. In further embodiments, protease inhibitors may be absent from the wash and elution buffers.

As noted above, the sample can be contacted with at least one protease inhibitor in an amount effective to reduce the level of protein A leachate in the purified antibody preparation relative to the level of protein A leachate present in the purified antibody preparation when the at least one protease inhibitor is not contacted with the sample. In some embodiments, the reduction of leachate can be at least 50%, or at least 75%, or at least 90%. The amount of reduction in leachate may be determined by measuring the amount of leachate in the purified antibody preparation, with or without the protease contacting step. Methods for performing such studies can be found in the Example section below.

In particular, it may be useful to determine the loading capacity of the support prior to developing a purification protocol. This can be accomplished by passing an antibody solution through a column of the support and measuring the UV absorbance (e.g., at 280 nm) of the effluent. Initially, the UV absorbance should be close to zero. As the support approaches saturation, the UV absorbance will increase until a plateau is reached, so that sample loading can be stopped. The maximum capacity can be determined by eluting the adsorbed antibodies from the support and calculating the amount of antibody that had been retained based on the UV absorbance of the collected antibody divided by the extinction coefficient. However, usually, sample loading is stopped soon after the UV absorbance of the effluent begins to increase above zero, to conserve sample.

The amount of reduction of leachate can be determined by measuring the level of protein A leachate as a faction of eluted antibodies without pre-treatment with protease inhibitor(s), and comparing this level to the level obtained after pre-treatment with protease inhibitor(s). For example, the amount of recovered (eluted) antibodies can be measured based on UV absorbance, and the amount of leachate can be measured using a protein A-specific immunoassay, for example. The choice and amount of protease inhibitors can then be adjusted to achieve the desired reduction of leachate.

It will be readily apparent to one of skill in the art that many other embodiments based on the above teachings are also possible, and the above teachings are not meant to be limiting in any way. Further the following non-limiting examples illustrate, but are not intended to limit, the present teachings.

### EXAMPLES

In the following examples, protein A chromatography was performed using a customized PerSeptive BioCad 700E HPLE system equipped with a stainless steel column (4.6 mm×10 cm) containing a bed of POROS® A50 resin (a protein A affinity support from Applied Biosystems).

Protein A Resin Capacity. The loading capacity of a protein A affinity support can be determined as follows. The column is equilibrated with 20 mM sodium phosphate, containing 0.15 M NaCl, pH 7.5 (equilibrium buffer, also called loading buffer). Human IgG from serum (Sigma Cat. No. G4386) is loaded at a concentration of about 5 mg IgG/mL, until the UV absorbance (280 nm) ceases to rise significantly. The IgG loaded support is then washed with equilibration buffer and then eluted with 75 mM acetic acid. Fractions of the 75 mM eluant are collected and then analyzed for antibody concentration. The column may then be washed with IM acetic acid and stored in 20% ethanol/equilibration buffer. A typical protocol is illustrated in Table 1 below.

TABLE 1

| | | | | | Col. | Linear | Purge (mL) |
|---|---|---|---|---|---|---|---|
| | | Flow rate | | Volume | Volumes | velocity | column |
| Step | Buffer | (mL/min) | Time | (mL) | (CV) | (cm/h) | offline |
| 01-A | Eq. Buffer | | 0.58 | | | | 10 |
| 01-B | Eq. Buffer | 1.38 | 6.65 | 33.24 | 20 | 498 | |
| 02-A | IgG (5 mg/mL) | | 1.08 | | | | 10 |
| 02-B | IgG (5 mg/mL) | 1.38 | 16.86 | 23.268 | 14 | 498 | |
| 02-C | Eq. Buffer | 5 | 7.6 | 24.93 | 15 | 1805 | |
| 03-A | 75 mM HOAc | 5 | 3.33 | 16.62 | 10 | 1805 | |
| 03-B | Eq. Buffer | 5 | 1.66 | 8.31 | 5 | 1805 | |
| 03-C | 1 M HOAc | 5 | 3.32 | 16.62 | 10 | 1805 | |
| 03-D | Eq. Buffer | 5 | 3.33 | 16.62 | 10 | 1805 | |

Antibody Purifcation. The POROS™ A50 column from the immediately preceding paragraph is equilibrated with equilibrium buffer. Antibody sample (human IgG from serum (Sigma Cat. No. G4386) is loaded at a concentration of about 5 mg IgG/mL solution in an amount sufficient to load the support with 15 to 20 mg IgG per mL of POROS A50 support. The IgG loaded POROS A50 is then washed with equilibration buffer and then eluted with 75 mM acetic acid. Fractions of the 75 mM acetic acid eluant are collected and then analyzed for the concentrations of IgG and Protein A. A typical protocol is illustrated in Table 2 below.

TABLE 2

| | | | | | Col. | Linear | Purge (mL) |
|---|---|---|---|---|---|---|---|
| | | Flow rate | | Volume | Volumes | velocity | column |
| Step | Buffer | (mL/min) | Time | (mL) | (CV) | (cm/h) | offline |
| 01-A | Eq. Buffer | | 0.58 | | | | 10 |
| 01-B | Eq. Buffer | 1.38 | 6.65 | 33.24 | 20 | 498 | |
| 02-A | IgG (5 mg/mL) | | 1.08 | | | | 10 |
| 02-B | IgG (5 mg/mL) | 1.38 | 4.82 | 6.648 | 4 | 498 | |
| 02-C | Eq. Buffer | 5 | 7.6 | 24.93 | 15 | 1805 | |
| 03-A | 75 mM HOAc | 5 | 3.33 | 16.62 | 10 | 1805 | |
| 03-B | Eq. Buffer | 5 | 1.66 | 8.31 | 5 | 1805 | |
| 03-C | 1 M HOAc | 5 | 3.32 | 16.62 | 10 | 1805 | |
| 03-D | Eq. Buffer | 5 | 3.33 | 16.62 | 10 | 1805 | |

Measurement of Leachate Reduction. Antibody purification was performed according to the protocol immediately above with five lots of human IgG in equilibrium buffer containing no protease inhibitors and with five lots of human IgG in equilibrium buffer containing 20 mM EDTA and 1 mg/mL Pefabloc™ SC. Protein A leachate concentrations were determined using a Protein A ELISA Kit from Repligen (Waltham, Mass.). IgG concentrations were determined by absorbance at 280 nm using an antibody extinction coefficient of 1.4. Results are shown in Tables 3A and 3B below, in which protein A leachate levels are expressed as ng protein A per mg IgG (parts per million, or ppm). As can be seen, leachate levels could be reduced by over 85%, and by over 90% in four out of five runs.

TABLE 3A

| | Protein A | IgG | Prot A/IgG |
|---|---|---|---|
| Run | (ng/mL) | (mg/mL) | (ng/mg) |
| 1 | 346 | 2.65 | 130.7 |
| 2 | 369 | 2.70 | 136.5 |
| 3 | 245 | 2.76 | 88.7 |
| 4 | 345 | 2.74 | 125.9 |
| 5 | 243 | 2.70 | 89.9 |

TABLE 3B

| | Protein A | IgG | Prot A/IgG | % Leachate |
|---|---|---|---|---|
| Run | (ng/mL) | (mg/mL) | (ng/mg) | Reduction |
| 1 | 33 | 2.671 | 12.2 | 90.6% |
| 2 | 24 | 2.721 | 8.7 | 93.6% |
| 3 | 32 | 2.700 | 11.9 | 86.6% |
| 4 | 24 | 2.707 | 9.0 | 92.8% |
| 5 | 9 | 2.621 | 3.4 | 96.2% |

Instead of Pefabloc™ SC, Calbiochem Protease Cocktail III (1:100 dilution) can also be used with good results.

Protease Quantification. Protease activity can also be detected or quantified using a suitable enzyme assay, such as a trypsin protease assay kit from Pierce. Human IgG (Sigma Cat. No. G4386) was found to contain approximately 50 ng trypsin activity/mg IgG.

All publications and patent applications mentioned herein are hereby incorporated by reference as if each publication or patent application was specifically and individually indicated to be incorporated by reference.

Although the invention has been described with reference to certain illustrative embodiments and examples, it will be appreciated that various modifications and variations can be made without departing from the scope and spirit of the invention.

1. A method of purifying an antibody sample comprising:
contacting the sample with a protein A affinity support under conditions such that antibodies are captured by binding to protein A on the support to form support-bound antibodies,
removing non-antibody components from the support bound antibodies, and
releasing the support bound antibodies from the support to obtain a purified antibody preparation,
wherein prior to or during said contacting, the sample is contacted with at least one protease inhibitor in an amount effective to reduce the level of protein A leachate in the purified antibody preparation relative to the level of protein A leachate that is present in the purified antibody preparation when the at least one protease inhibitor is not contacted with the sample.

2. The method of claim 1, wherein the at least one protease inhibitor comprises a metalloproteinase inhibitor.

3. The method of claim 2, wherein the at least one protease inhibitor comprises a metal chelator.

4. The method of claim 2, wherein the at least one protease inhibitor comprises ethylenediamine tetraacetic acid (EDTA).

5. The method of claim 1, wherein the at least one protease inhibitor comprises a serine protease inhibitor.

6. The method of claim 1, wherein the at least one protease inhibitor comprises an inhibitor of at least one of trypsin, chymotrypsin, plasmin, plasma kallikrein, thrombin, clotting factors, tissue proteinases, leukocytic proteinases, elastase-like serine protease and urokinase.

7. The method of claim 1, wherein the at least one protease inhibitor comprises an inhibitor of at least one of trypsin, chymotrypsin, plasmin, plasma kallikrein and thrombin.

8. The method of claim 1, wherein the at least one protease inhibitor comprises a benzenesulfonyl fluoride compound.

9. The method of claim 1, wherein the at least one protease inhibitor comprises at least two different serine protease inhibitors.

10. The method of claim 9, wherein the at least two different serine protease inhibitors are inhibitors of at least two of trypsin, chymotrypsin, plasmin, plasma kallikrein, thrombin, clotting factors, tissue proteinases, leukocytic proteinases, elastase-like serine protease and urokinase.

11. The method of claim 9, wherein the at least two different serine protease inhibitors are inhibitors of at least two of trypsin, chymotrypsin, plasmin, plasma kallikrein and thrombin.

12. The method of claim 5, wherein the at least one protease inhibitor comprises a metalloproteinase inhibitor.

13. The method of claim 5, wherein the at least one protease inhibitor comprises a metal chelator.

14. The method of claim 5, wherein the at least one protease inhibitor comprises ethylenediamine tetraacetic acid (EDTA).

15. The method of claim 1, wherein the at least one protease inhibitor is provided in an amount effective to reduce the level of protein A leachate in the purified antibody preparation by at least 50% relative to the level of protein A leachate that is present in the purified antibody preparation when the at least one protease inhibitor is not contacted with the sample.

16. The method of claim 1, wherein the at least one protease inhibitor is provided in an amount effective to reduce the level of protein A leachate in the purified antibody preparation by at least 75% relative to the level of protein A leachate that is present in the purified antibody preparation when the at least one protease inhibitor is not contacted with the sample.

17. The method of claim 1, wherein the at least one protease inhibitor is provided in an amount effective to reduce the level of protein A leachate in the purified antibody preparation by at least 90% relative to the level of protein A leachate that is present in the purified antibody preparation when the at least one protease inhibitor is not contacted with the sample.

18. The method of claim 1, wherein said protein A affinity support is provided in a chromatography column, and said removing comprises passing a buffer through the support under conditions such that support bound antibodies are retained on the support.

19. The method of claim 1, wherein the sample comprises a monoclonal antibody or monoclonal antibody fragment.

20. The method of claim 1, wherein the sample comprises a polyclonal antibody or polyclonal antibody fragment.

21. The method of claim 1, wherein the sample comprises an IgG antibody or IgG antibody fragment.

22. The method of claim 1, wherein the sample comprises a human antibody or human antibody fragment.

23. The method of claim 1, wherein the sample comprises a human IgG antibody or human IgG antibody fragment.

24. The method of claim 1, wherein the sample comprises serum or ascites or is obtained from serum, ascites, or tissue culture.

25. The method of claim 1, wherein the sample comprises or is derived from human blood.

26. The method of claim 1, wherein the said releasing comprises eluting the purified antibody preparation with an aqueous solution comprising acetic acid.

27. The method of claim 1, wherein following said release, the purified antibody preparation is neutralized with a neutralization buffer.
